(19) United States
(12) Patent Application Publication
(10) Pub. No.: US 2012/0259227 A1
(43) Pub. Date: Oct. 11, 2012

(54) DETECTION METHOD

(76) Inventors: Allan Walter Wilson, Hamilton (NZ); Vicki Clare Farr, Katikati (NZ); Richard John Doohan, Hamilton (NZ)

(21) Appl. No.: 13/518,272

(22) PCT Filed: Dec. 21, 2010
(86) PCT No.: PCT/NZ2010/000254

§ 371 Date: Jun. 21, 2012

(30) Foreign Application Priority Data
Dec. 24, 2009 (NZ) 582407

(51) Int. Cl. A61B 5/01; A61D 99/00; A61B 6/00; A61B 10/00

(52) U.S. Cl. 600/474; 600/549

(57) ABSTRACT

A method is provided for operating an apparatus for detecting if oestrus is imminent or present in a milking animal, the apparatus configured to be operated by the steps of:
a) defining a processing area which encompasses two or more independent heat outputs including a portion of the rear of the milking animal;
b) measuring an indicator of heat output from that area with at least one sensor; and
c) using measurement obtained in b) to determine if an oestrus condition is imminent or present.

### TECHNICAL FIELD

This invention relates to a detection method.

In particular, the present invention relates to a method of detecting oestrus in a milking animal such as a cow.

### BACKGROUND ART

The dairy industry is dependent on the oestrus cycle of the cow. Most modern farming techniques use artificial insemination of a cow to increase the value of bloodlines. This is a skilled procedure usually requiring a trained technician to visit the farm with multiple straws of semen stored in liquid nitrogen. The timing of when the cow is inseminated is critical. If the semen is administered at the wrong time of the cow's oestrus cycle, then a successful insemination will not occur as it takes some time to determine whether a cow has been successfully inseminated, and considerable time can be wasted before the cow is inseminated again.

Further, the cow usually is only in oestrus over a 6-24 hour period averaging 15 hours once every 18 to 24 days. So it can be easy to miss the oestrus period.

In many instances, a cow that is not successfully inseminated is culled at the end of the current lactation (as they would need to be carried for two winters and a milking season) which is naturally undesirable and also highly expensive to the farmer.

Another disadvantage of unsuccessful attempts of insemination is that multiple visits from the technician are required, which can be expensive—both in terms of the technician's time and the waste of semen.

Thus, numerous attempts have been made to develop and accurate method for determining the occurrence of oestrus in cows.

Secondary signs of standing oestrus can be observed through farmer observation of the following:
Congregating. Cattle that are in standing oestrus naturally seek out other animals in oestrus and form a small group, referred to as the sexually active group. They make physical contact with each other, standing head to tail, circling, butting heads, and resting their chins on the back or hip of other cows/heifers.
Whenever a small group of animals gathers together, it should be watched closely for animals in standing oestrus.
Mounting other animals. Animals in standing oestrus or approaching this stage will usually try to mount other animals.
Nervousness or restlessness. This may be excessive walking and bawling. Any animal that is moving when other animals are relatively stationary or walking a fence line in search of a bull.
Roughed up tailhead. Normally, the hair on the tailhead lies down and points toward the tail, but the hair on the tailhead of an animal that has been ridden may be roughed up to the point where it sticks almost straight up. A cow/heifer that has been ridden hard may sometimes have the hair rubbed off of her tailhead.
In muddy areas, mud will often be plastered on both flanks and sometimes up along the back and ribs.
However, both sides of the animal must show signs of being ridden, since an animal cannot be ridden and marked on only one side.
Swollen vulva. A moist, red, and swollen vulva is often associated with standing oestrus. However, this can be difficult to determine and may be of limited value.
Bloody mucus from vagina. Two to three days after standing oestrus a bloody discharge from the vulva may be observed. This is normal and only means that the animal was in standing oestrus earlier. It is too late to inseminate the animal, but it is an indicator that she should be monitored for standing oestrus in 17 to 21 days.

However all secondary signs require significant observational skill and thus accuracy is limited.

Commercial aids have been developed to assist detection.

Most commonly, these aids involve the detection of mounting by other cows—either by marking or detection of irritation. For example, cows are often tail painted which is a laborious manual task performed by the farmer. When a cow is mounted, the tail paint is rubbed off, indicating that the cow is in oestrus. This still requires manual detection of the condition of the tail paint by the farmer.

While it is a relatively inexpensive process this is labour intensive and requires some skill to accurately interpret the symptoms.

A product sold under the trade mark Kamar™ is an adhesive patch with a reservoir of dye. The patch is placed on the rump of the cow such that when the cow is mounted, the reservoir is ruptured giving the visual indication. These patches are more expensive than tail paint (although perhaps slightly less labour intensive to apply), and unfortunately are an environmentally hazard being a disposable product.

Pedometers are also used on cows as an increased movement is often an indication of oestrus. However, this is also a disposable product and relatively expensive.

It should be appreciated that these days herd numbers in the many hundreds and therefore to apply any indicator on an individual basis leads to expense, time and environmental hazards when disposable items are used.

Even in herds using a selection of these detection aids as well as visual observation for behavioural symptoms, at least 5% of cows will not be correctly diagnosed in oestrus at the correct time. In less well managed herds, this can increase to over 40%, especially in large feedlots or in small herds of less than 20 cows where they are continually housed.

Vasectomised bulls are sometimes fitted with marking devices such as a chin-ball marker which leaves a mark after mounting a cow. However marks can also be left through the animal just rubbing its chin.

In order to address these problems, intravaginal devices have been developed such as the CIDR™ which is a silicon device impregnated with progesterone which helps regulate the cows' cycle so that the timing of oestrus can be predicted more accurately.

While effective, it can be time consuming and expensive to fit CIDRs into each individual cow in large herds.

Other breeding programmes which utilise oestrus inducing fertility drugs are highly dependant on the farmer or technician administering the treatment at precise intervals. Often, a stage of treatment is missed because a cow could not be located or the farmer is pre-occupied, thus reducing the effectiveness of the treatment. Non compliance rates can be as high as 30% in some herds.

An investigation of skin temperature differentials in relation to oestrus in dairy cattle was conducted by Hurnik (Reference: Hurnik et al, J Anim Sci 1985. 61:1095-1102).

The researcher was very careful to ensure that clear thermal images were obtained. This included many manual adjustments such as the cows tail held out of the way, the cow dried and waste matter removed. Naturally any method that required all of these manual adjustments would be impractical on a commercial scale particularly given the large number of herds for which the desired to have automatic detection.

Hurnik's experiments involved taking images of just the gluteal regions of barn cows in stalls and then manually counting pixels on photographs taken to obtain an indication of heat output.

While Hurnik identified that the temperature in the gluteal region of the cows body tends to increase, he noted that the frequency of false positives and false negatives would render the technique of limited use for routine oestrus detection. A reason for this conclusion was the inability to account for environmental conditions.

Thus, while interesting, Hurnik's research did not provide a reliable and practical method of detecting oestrus.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DISCLOSURE OF THE INVENTION

According to one aspect of the present invention there is provided an apparatus for detecting if oestrus is imminent or present in a milking animal,

the apparatus configured to be operated by the steps of
a) defining a processing area which encompasses two or more independent heat outputs including a portion of the rear of the milking animal, and
b) measuring an indicator of heat outputs from that area with at least one sensor, and
c) using the measurement obtained in b) to determine if an oestrus condition is imminent or present.

According to another aspect of the present invention there is provided an apparatus for detecting if oestrus is imminent or present in a milking animal, the apparatus to be configured to be operated by the steps of
a) defining a processing area including a portion of the milking animal which can be subject to physical interaction during oestrus, and
b) measuring an indicator of heat output from that area with at least one sensor, and
c) using the measurement obtained in b) to determine if oestrus condition is imminent or present.

In preferred embodiments of the present invention, the milking animal is a cow. However, it should be appreciated that the present invention could apply to other milking animals such as goats and sheep.

The present invention can be used to determine a standing oestrus condition which is defined as the first occasion that a cow is mounted and stands. However, as can be seen in the following description, the present invention can be used to determine pro-oestrus, that is, when oestrus in imminent. This is significant given the narrow window that a cow is available for successful insemination.

This could increase conception rate by catching cows at onset, rather than being too late after the observation of standing oestrus.

By detecting the appropriate condition at morning and afternoon milkings, cows can be drafted for mating appropriately.

Defining a processing area is an important step in the present invention.

In the past, scientists have used instruments in the field that targeting a spot on the animal to gain a temperature reading, or have applied isotherm techniques to the gluteal region of an animal This is because some researchers believe the highest point temperature on an animal is an indicator of core temperature and therefore can give an indication as to whether oestrus is occurring.

However, these methods are highly inaccurate and can require many measurements in order to determine the likely highest temperature.

At least a portion of the rear of the milking animal may be included in the processing area as this has been identified as being the most receptive to changes associated with increased blood supply in the oestrus condition.

One aspect of the present invention is the provision of two or more independent heat outputs which gives greater accuracy in the detection of oestrus. These heat outputs could arise from the back, tail crest, pin bones as well as the vulva region.

The inclusion of these additional areas and the processing area means that there is less chance of false negative or positives occurring.

In particular, it is important to note that with animals which are not barn animals (such as those farmed in Australasia), it is more likely for the cows to exhibit riding behaviour. Thus, in some instances of the present invention the heat output detected may only be that associated with riding behaviour. This requires careful selection of the processing area in order to capture those areas of rubbing and lesions that occur as a consequence.

In preferred embodiments of the present invention, the present invention is conducted within a milking system and therefore the processing area can be well defined as being in the region of the rear of a milking stall.

Taking measurements within a milking shed is an important feature of the present invention. The milking shed offers conditions by which the animals are contained within a defined area as opposed to taking measurements out in the field.

By using the milking shed, and particularly a milking stall, sensors can be set up in a permanent or semi permanent position with one sensor being able to take readings from many cows as they enter the milking stall with which the sensor is associated. It may be that only a single sensor is used that is mounted with respect to a point that the animal passes while being milked or on its way to be milked—or as it exits the milking parlour.

It can be seen therefore that the choice of where to mount the sensor is very important and can provide considerable advantages when compared to having individual detection devices associated with each animal.

Preferably, the processing area will include the external pudendum.

In a preferred embodiment, the vulva will be an area included in the processing area. The vulva is defined as the area within the pin bones of the cow below the top of the tail head. Its heat output is generally a reflection of a hormonal response.

In some embodiments of the present invention, at least part of the pin bones of the cow is also included within the processing area as one of the heat outputs. This is because recent mounting of the animal by others (that is, a standing oestrus condition) can inflame the pin bones giving off a heat signature indicating an oestrus condition.

Preferably the tail ridge and surrounds are included along with the top of the tail head.

In some embodiments, the sensor may be mounted above the cow and angled such that a greater proportion of the pin bone area is captured by the sensor in addition to the vulva or external pudendum.

The inventors have determined that an area can be selected which is greater than the rear of the milking animal, provided suitable data filtering is applied to remove the effects of the ambient environment, milking equipment and the like.

By selecting a greater area, movement of the cow within the milking stall can be accounted for ensuring that all of the relevant parts of the cow that is required to be measured are captured within the processing area.

The present invention can be used with many different types of indicators of heat outputs from the processing area.

In one embodiment of the present invention; a thermographic camera may be used on the processing area. Again, it should be appreciated that the processing area may include areas outside of the external pudendum and pin bones including part of the milking shed environment.

To limit the effects of non essential heat sources, one embodiment of the present invention applies a filter to the images received by the thermographic camera such that all heat sources below a particular temperature threshold are ignored.

The threshold can vary according to the particular environment.

In one embodiment of the present invention, an absolute threshold is chosen, say 36° C. as the normal cows internal temperature is around 38.4° C.

An alternative threshold would be to determine a maximum temperature on a cow and subtract certain number of degrees from that (say 2-4° C.).

Another method could be to obtain historical temperature readings of a cow and use as a subsequent threshold only those temperatures above the average of the cow when it is known not to be in an oestrus condition.

It should also be appreciated that the independent heat outputs being measured can have different thresholds to each other.

Since the oestrus condition can be expected to occur every 18 to 24 days in cows that are not pregnant and have recovered from a recent calving, daily monitoring over an extended period preceding and following a first insemination can provide a source of reference to enhance the probability of correctly diagnosing the oestrus condition.

It may be that for less temperate climates that the best time to read the cows would be with the morning milking as there is less likelihood that the external surface of the cow would have been warmed through exposure to the sun—in comparison with afternoon milking. However, with the use of herd normalization the effects of ambient conditions can be minimised thus allowing oestrus detection at any time.

Once the threshold has been applied, then a number of different methods could be used to assess the relevance of the images obtained.

For example, all pixels above the threshold used may be counted to give an absolute reading. Any number of counts above a certain amount may be an indicator of oestrus.

For example, the amount may be a certain percentage above the “average” historical temperature of that cow not in oestrus.

Alternatively, the count may be just an absolute number above which any cows considered to be in oestrus.

Alternatively, it has been recognised that oestrus is a condition which has a gradual onset over a period of several hours and is associated with increasing concentrations of oestrogen being secreted by the ovary up until the time of ovulation when oestrogen synthesis ceases. Thus, a sharp change from one day to the next in gradient between historical temperatures to the present temperature may be an oestrus indicator.

The inventors have found that by using the above methods, the jump between the oestrus state and the non-oestrus state is comparatively large. Thus, the present invention can be used with a low resolution camera.

An alternate embodiment of the present invention could use a fairly simple sensor such as a non-contact thermopile sensor to measure heat flux from the processing area.

In one embodiment, the thermopile may be combined with another sensor (e.g. optical) to track the processing area and measure total heat flux coming from that area.

Similar mathematical methods can be used to determine whether the heat flux per area is an indicator of oestrus as described with reference to the embodiment using a camera.

It should be appreciated that the timing of the increase in heat from the outputs will most likely differ. For example, it is expected that the increase in heat from the vulva region. (hormonal response) could occur a day before additional heat is detected from a rubbing response. Thus the vulva reading could indicate an imminent oestrus condition and the latter pin bone reading could confirm oestrus.

One of the many advantages of the present invention is that all of the calculations can be made before the cow enters the drafting race. Thus, the farmer can get feedback around the time of milking as to whether the cow is in oestrus or about to be. Therefore, insemination can be planned for the right time.

In preferred embodiments of the present invention, herd normalisation may be used in order to give greater elimination of environmental effects. For example, each cow may be compared with the temperature of say 10 cows before and after in the milking/drafting process. Thus, the average herd temperature can be used as a base to compare individual cows against. Therefore a spike in temperature arising from oestrus against the herd average would be more accurate than taking an absolute temperature which may be affected by environmental conditions—such as a hot day.

This overcomes one of the limitations observed by Hurnik.

In preferred embodiments, the present invention is used with the cow's individual ID and database with its individual data set histories. Thus, the accuracy (sensitivity and specificity) of the heat output can be improved. For example, milk yield and/or rumination data may provide additional indicators of oestrus conditions.

In some embodiments of the present invention there may even be provided automatic drafting of the animals which are detected to be in oestrus. That way the animals can be separated from the rest of the herd for insemination by the technician.

The present invention has considerable advantages over the prior art.

As can be seen in the Best Modes section, the statistical significance of readings associated with the present invention means that highly accurate detection of oestrus can occur.

The present invention is particularly suitable for farming systems whereby the animals are in a position to exhibit riding behaviour (such as in barns) as this allows a further input to give an indication of the oestrus condition.

Early detection is also possible leading to increased odds of conception.

The present invention does not require a high degree of labour, particularly as the elimination of environmental conditions and the use of additional data means that automatic sensing can occur without the manual manipulations employed by Hurnik.

There are no environmental concerns as there are no disposable detection systems used.

With rotary and robotic milking sheds, only one detection system is required per shed, making overall the cost of the present invention comparatively cheap on a per cow basis. With herringbone sheds cows usually have to enter/exit in single file so a single sensor could be placed appropriately.

The present invention also leads to the ability to auto draft cows needing insemination.

Farmers tend to be more conservative in diagnosing oestrus in inseminated cows as distinct from cows yet to be inseminated. The presumption is that in the former case, the cow may be pregnant. To inseminate again when it is pregnant but not in oestrus can disrupt the pregnancy. Even if that does not occur, the straw of semen has been wasted.

An advantage with the proposed sensor is that it represents a form of objective assessment independent of any human influence, and it can instantly recall previous history of relevance. For example, heat dates and/or insemination dates. Therefore, the use of the sensor can reduce the average interval from first insemination to conception in a herd by minimising the incidence of missed oestrus events that may occur after the first (or subsequent) insemination.

Additional uses of the sensor can occur in the period after an artificial insemination program has concluded.

In New Zealand herds it is almost standard practice to allow bulls to graze with the herd to impregnate those cows that have failed to conceive to artificial insemination. It is often difficult to accurately identify these cows and this necessitates pregnancy testing to confirm pregnancy status and conception date by estimation.

The use of the sensor will identify which cows are served by a bull by date as well as identifying those that persist with failing to conceive. Those cows that have failed to conceive or loss a conceptus (abort) will be detected because they will still be experiencing oestrus events. These are the cows that are most likely to be culled from the herd sooner than pregnant herd mates. The cost of pregnancy testing will be dramatically reduced in most herds.

### BRIEF DESCRIPTION OF DRAWINGS

Further aspects of the present invention will become apparent from the following description which is given by way of example only and with reference to the accompanying drawings in which:

FIG. 1 is a thermographic image of a cow not entering oestrus, and

FIG. 2 is a thermographic image of a cow entering oestrus, and

FIG. 3 shows a threshold area count of both the vulva and tail ridge regions of a cow indicating oestrus, and

FIG. 4 shows the threshold area count of the vulva area of two cows during oestrus and that of a single anoestrous cow

FIG. 5 shows the threshold area count of the tail ridge area of two cows during oestrus and that of a single anoestrous cow

FIG. 6 shows how there is a corresponding drop in progesterone with oestrus as detected by the present invention, and

FIG. 7 is an algorithm illustrating one method of operating the apparatus in accordance with your present invention.

### BEST MODES FOR CARRYING OUT THE INVENTION

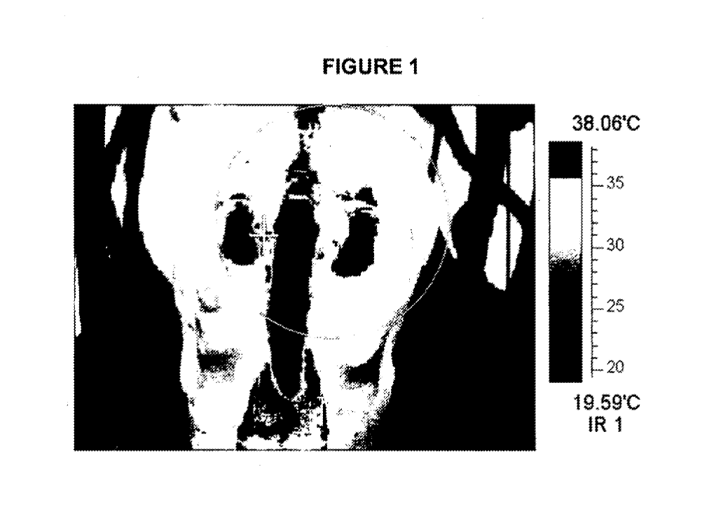

FIG. 1 represents a thermographic image of a cow which is not in oestrus nor about to enter oestrus. As can be seen, the processing area shown by a circle has very little data points recorded above 35° C. However, below the processing area is the udder of the cow which has a number of data points above the 35° C. threshold. Thus, it can be seen that it is important to chose an appropriate processing area that does not include the udder.

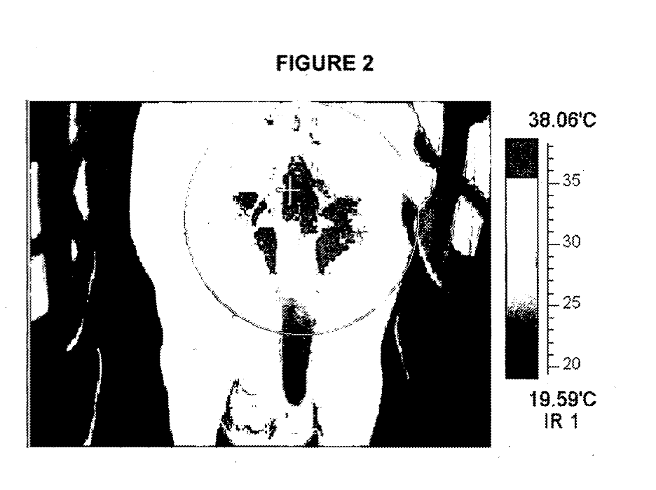

In contrast, FIG. 2 illustrates a cow which is about to enter oestrus. In this image, the processing area includes a number of data points above 35° C.

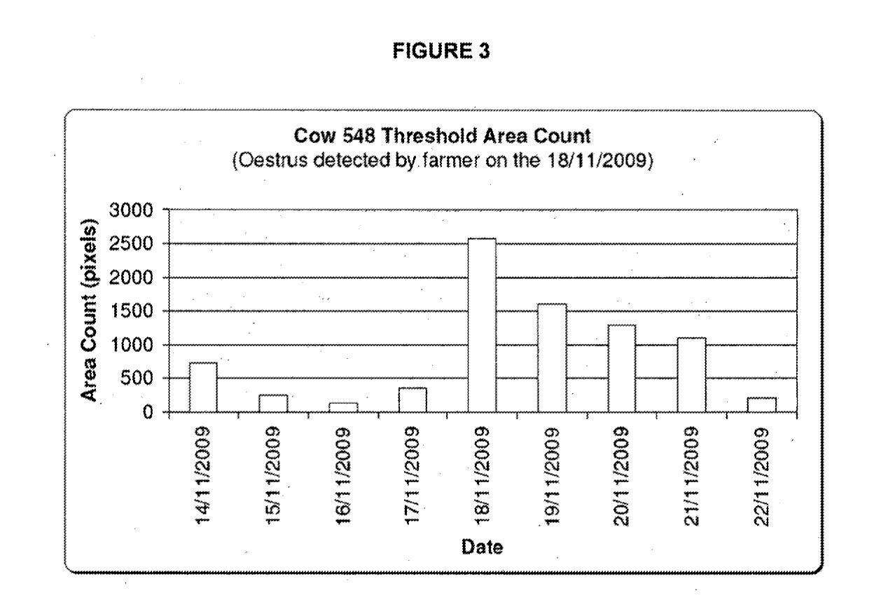

FIG. 3 is a graphical representation of the count of data points above a threshold within a processing area such as that illustrated in FIGS. 1 and 2.

As can be seen from FIG. 3, the farmer detected oestrus in this particular cow. Quite dramatically, the area count on the cow has risen from in the order of 400 pixels to over 2500 pixels. Such a dramatic increase makes the detection of oestrus a very easy and obvious task in accordance with the present invention.

Below is a description of the experimental trials which illustrate in greater detail how the present invention can be performed.

Experiments:

The camera and thermopile sensor were installed above ‘cups on’ on a rotary platform and images taken of all cows during every milking. Images were automatically matched to cow identity so that data could then be combined with other cow information. It was necessary to capture data early in the milking, so that it would be possible for cows to be drafted for mating as they left the platform. The position and angle of the cameras allowed image capture of both the vulva, and the hip bones and tail ridge areas, where most of the rubbing and often abrasions occur. The camera was also out of the way of the milking staff and not as likely to be knocked or hosed.

Examples of the recorded images from the initial trial are shown in FIGS. 1 and 2. To determine which and when cows were in oestrous the farmer monitored tail paint routinely at every morning milking during mating. As an alternative, and less subjective reference method, progesterone profiles were constructed by collecting milk samples every seven days over the mating period for milk progesterone analyses in the second trial.

Results and Discussion

The images and data presented are taken from during the mating period, including only the morning milkings when the farmer was manually selecting and drafting cows for mating. Results suggest that data from afternoon milkings is also valid. Examples, from the first trial, of total output from the vulva and tail ridge region are shown in FIGS. 1, 2 and 3. The inventors have since found that the temperature differentials indicating oestrus are different for different heat output areas.

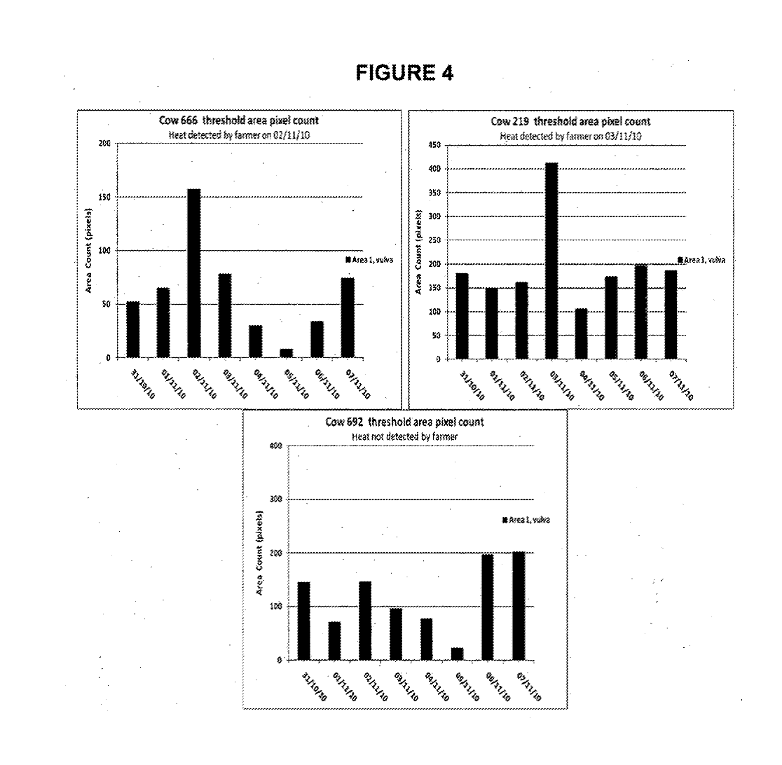

For example, as can be seen by the graph in FIG. 4, the increase in heat in the ‘vulva’ region, i.e. the area between the pin bones and surrounding the top of the tail, but below the top of the tail ridge, indicates an imminent oestrus condition.

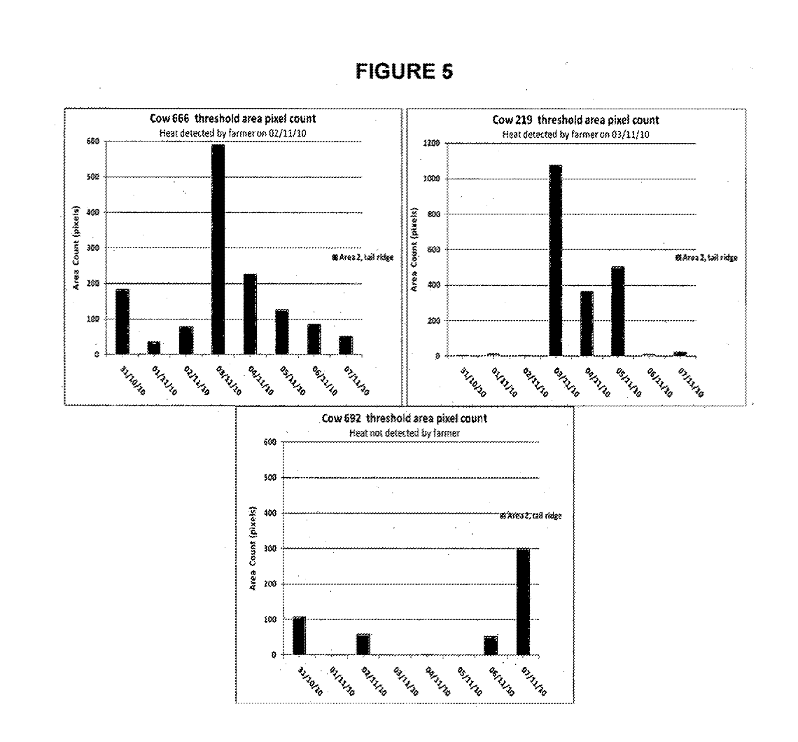

Likewise, the graph in FIG. 5 illustrates the increase in heat in the ‘tail ridge’ region, i.e. the sacral spinal ridge beyond the top of the tail extending forward until the line of the hip bones. This increase could be as a result of increased blood flow to the area due to rubbing or abrasions from riding and/or hormonal changes indicating an oestrus condition.

Differentiation between the heat output from the vulva area as a result of hormonal changes; to that from the tail ridge area, which could be as a result of both hormonal changes and a physical or rubbing response; is more accurate than just a heat output from the entire area.

Traditionally a farmer would usually only be able to detect cows during, or after oestrous has occurred using only riding behaviour or tail paint observations at the morning milking. Furthermore, this method will only detect those cows that are being ridden.

Because cows are generally milked only 1-3 times per day the interval between image capture can vary, sometimes exceeding 16 hrs. Therefore the timing of image capture will vary in relation to the hormonal responses during oestrous. Individual cow behaviour before or during oestrous and her proximity to herd mates, in particular sexually active groups, will also affect the relationship between the timing and degree of change in outputs from the designated areas.

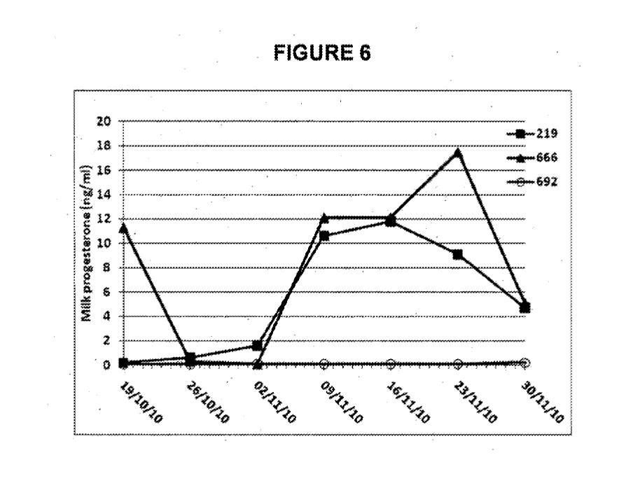

Hence, some images may capture increases in thermal output from both areas concurrently (cow 219, in FIGS. 5 and 6); while in others the vulva increase may precede that in the tail ridge (cow 666, in FIGS. 5 and 6); and in some there may only be an increase in either the vulva or the tail region during oestrous. The ability to combine and compare the timing and extent of these changes gives more weight to the detection decision.

To confirm the accuracy of these results, the profile of milk progesterone concentration, which reflects the concentrations in blood, is shown in the graph in FIG. 6. Milk progesterone concentration for the two cows during an oestrous cycle are compared to the non-cycling or anoestrous cow over the same period. The dramatic fall in milk progesterone precedes the onset of oestrous and corresponds to the subsequent oestrus condition detected through the present invention.

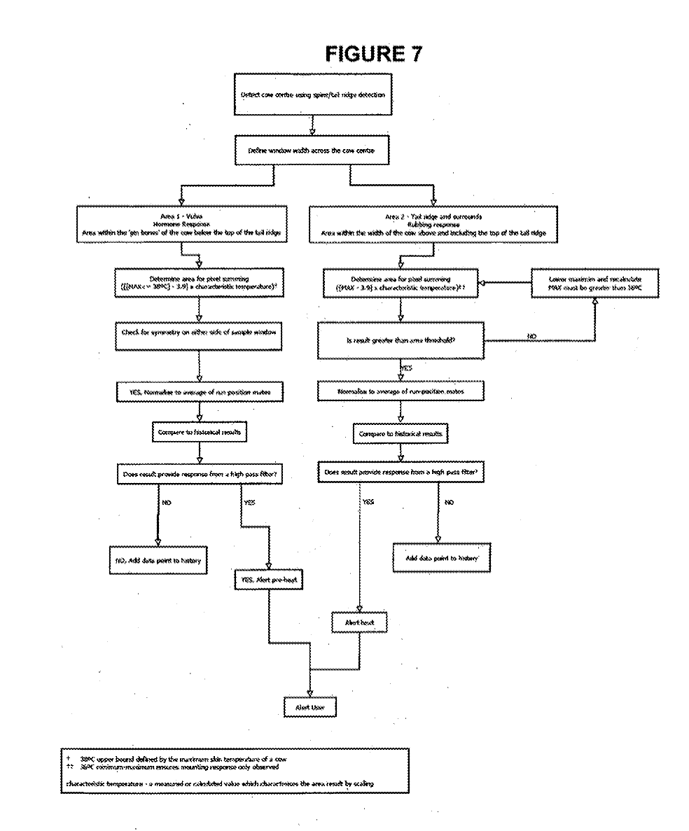

Finally, FIG. 7 illustrates a potential algorithm that can be used to operate apparatus in accordance with the present invention.

The algorithm in FIG. 7 is fairly self explanatory, but key points should be noted.

Firstly, a single window encompassing the independent heat outputs is defined.

Secondly, the two output areas have differing calculations to determine the likelihood of oestrus.

Thirdly, historical results are used in relation to both areas, and the data obtained is returned to the historical record. Thus, this iterative process can provide more data leading to better normalisation.

Finally, it is with positive indicators of imminent or actual oestrus from one or both areas that the user is alerted of a oestrus condition. If output increases occur in the tail ridge region alone we are at least as good as the traditional methods of ‘rub’ or ‘riding’ detection.

If we also detect an increase in the vulva area, either before or coincidentally with the tail ridge, it adds weight to the alert. Increases in the vulva area alone may be able to detect those cows that show less obvious symptoms of behavioural oestrous.

Aspects of the present invention have been described by way of example only and it should be appreciated that modifications and additions may be made thereto without departing from the scope thereof as defined in the appended claims.

1. A method of operating an apparatus for detecting if oestrus is imminent or present in a milking animal,
the apparatus configured to be operated by the steps of:
a) defining a processing area which encompasses two or more independent temperature outputs including a portion of the rear of the milking animal,
b) measuring the temperature outputs from that area with at least one sensor remote from the animal, and
c) using a measurement obtained in b) to determine if an oestrus condition is imminent or present.

2. The method as claimed in claim 1, wherein the processing area includes a portion of the milking animal which can be subject to physical interaction during oestrus, and wherein one independent temperature output is as a consequence of physical interaction with that portion of the animal.

3. The method as claimed in claim 1 wherein the sensor is a thermographic camera.

4. The method as claimed in claim 1 wherein the sensor is a thermopile.

5. An apparatus for detecting if oestrus is imminent or present in a milking animal
configured for performing the method of claim 1, and wherein the sensor is one of a thermographic camera and a thermopile, and where the processing area includes a portion of the milking animal which can be subject to physical interaction during oestrus, and wherein one independent temperature output is as a consequence of physical interaction with that portion of the animal.

6. The method as claimed in claim 1 wherein the milking animal is a cow.

7. The method as claimed in claim 1 wherein the processing area is in the region of the rear of a milking stall.

8. The method as claimed in claim 1 wherein the processing area includes the vulva.

9. The method as claimed in claim 1 wherein the processing area includes a tail ridge and surrounds of the milking animal.

10. The method as claimed in claim 1 further including applying a filter to images received by the sensor.

11. The method as claimed in claim 10 wherein all pixels above a filter level are counted to give a reading.

12. The method as claimed in claim 2 wherein the indicator of oestrus condition is a change in temperature gradient over time.

13. The method as claimed in claim 1 which uses historical cow data in combination with the measurements taken in accordance with the method.

14. The method as claimed in claim 1 wherein the method is applied during morning milking.

15. The method as claimed in claim 1 further including using herd normalisation of temperatures.

16. An apparatus programmed with a computer program configured to be executable by a processor to perform the method of claim 1.

17. A computer program configured to be executable by a processor to perform the method including the steps as claimed in claim 1.

18. (canceled)

19. (canceled)
